(19) United States
(12) Patent Application Publication
(10) Pub. No.: US 2006/0051392 A1
(43) Pub. Date: Mar. 9, 2006

(54) Porous coatings for drug release from medical devices

(75) Inventors: Kenneth T. Heruth, Edina, MN (US); Edouard Koullick, Golden Valley, MN (US); Mark S. Lent, Brooklyn Park, MN (US)

Correspondence Address: MEDTRONIC, INC., 710 MEDTRONIC PARK, MINNEAPOLIS, MN, 55432-9924, US

(73) Assignee: Medtronic, Inc., Minneapolis, MN (US)

(21) Appl. No.: 10/934,216

(22) Filed: Sep. 3, 2004

(51) Int. Cl. A61F 2/00

(52) U.S. Cl. 424/423

(57) ABSTRACT

Extravascular implantable medical devices are described. The devices include a polymeric layer comprising a polymeric matrix and pores. Therapeutic agent is loaded in the matrix, in the pores, or in the matrix and the pores. The devices include a structural surface layer. Additional therapeutic agent may be loaded in or on the surface layer. The devices may also include one or more intermediate layer, into or onto which additional therapeutic agent may be loaded.

### FIELD

The present disclosure relates to medical devices coated with porous polymers as vehicles for drug delivery.

### BACKGROUND

Implantation of medical devices, such as pacemakers, neurostimulators, implanted drug pumps, leads, catheters, etc, has been associated with adverse consequences, such as formation of scar tissue surrounding the implant, infection due to bacteria introduced during implantation, and tissue proliferation in blood vessels after a stent implantation. Attempts to prevent or control such adverse reactions have included administration of drugs, completely separate from the intended primary therapy of the implanted medical device. In some cases, systemically administered drugs, e.g. orally, intravenously, or intramuscularly administered drugs, have proven effective in treating complications due to medical device implantation. In other cases, systemic delivery has been ineffective due to, e.g., pharmacokinetic or pharmacodynamic characteristics of the drug, the location of the implanted device, or side effects of the drug. To increase effectiveness in these situations, some implanted devices have been modified to elute the drug into the surrounding tissues.

One common way of providing local drug elution is to dispose a polymer layer on the implantable medical device and embed the drug into the polymer during manufacturing. When hydrated after implant, the drug diffuses out of the polymer into surrounding tissue. Various methods of impregnating polymers with drugs have been used, including mixing the drug into the melted polymer prior to processing (e.g. molding or extrusion), and diffusing the drug into a finished polymer component using chemicals to swell the polymer for rapid loading. In some cases, the implantable medical device (IMD) is made from a polymer that is compatible with the drug, and the drug can be loaded directly into the device. However, many IMDs are made from metals or from polymers that are inherently incompatible with the desired drug. In such situations, the IMD can be coated with a thin layer of a compatible polymer, and the drug can be loaded into the coating layer.

However, problems exist with current loading technology. For example, it can difficult to load large quantities of drugs or to adjust release rates when conventional biomaterials, such as silicone rubber and polyurethane, are used as a matrix for drug loading.

A good deal of effort in this area has been focused on drug-eluting intravascular medical devices, such as stents and balloon catheters. Localized intravascular delivery of drugs, such as that achievable by drug-eluting intravascular devices, presents unique challenges. For example, fluid, such as blood, can rapidly carry drug away from the desired local delivery site. One proposed method of increasing the loading of intravascular drug-eluting devicrs includes electrophoretically loading a porous polymer coating of the intravascular medical device. The electrophoretic method apparently allows for increased drug loading. Another methods suggests the repeated exposure of a porous polymer coated device to a saturated solution of drug. By repeated exposure and drying, a larger quantity of drug may be loaded in the porous polymer.

Difficulties associated with drug-eluting extravascular implantable medical devices have not been adequately addressed.

### BRIEF SUMMARY

In an embodiment, the invention provides an extravascular implantable medical device. The devices comprise a polymeric layer comprising a polymeric matrix and pores. Therapeutic agent is loaded in the matrix, in the pores, or in the matrix and the pores. The device may further comprise a structural surface layer. Additional therapeutic agent may be loaded in or on the surface layer. The device may also further comprise one or more intermediate layer, into or onto which additional therapeutic agent may be loaded.

Such a device may provide one or more advantages over existing non-vascular medical devices. For example, pores in the polymeric layer increase the rate at which therapeutic agent may be released from the matrix. Further, loading therapeutic agent in the pores, as opposed to just the matrix, can increase the total amount of therapeutic agent that may be loaded into the device. In addition, therapeutic agent loaded into the pores will be quickly released from the device after implantation. Loading therapeutic agent into or on the surface layer and/or one or more intermediate layers allows for additional loading capacity, as well as finer control of the release profile of therapeutic agent from the device. Another advantage of a polymeric layer comprising pores is the ability of tissue to integrate with the pores after implantation. Thus, release of therapeutic agent may become more effective as less drug is removed into interstitial fluids, surrounding tissue, etc. These and other advantages will become evident to one of skill in the art upon reading the disclosure herein.

### BRIEF DESCRIPTION OF THE DRAWINGS

FIG. 1 is a diagrammatic illustration of a neurostimulatory system implanted in a patient.

FIG. 2 is a diagrammatic illustration of an infusion pump system implanted in a patient.

FIG. 3A is a diagrammatic illustration of a cross section of a portion of a device comprising a surface layer and polymeric layer comprising pores and therapeutic agent disposed in the pores, the polymeric layer being disposed on or about the surface layer.

FIG. 3B is a diagrammatic illustration of a cross section of a portion of a device comprising a surface layer and polymeric layer comprising therapeutic agent and pores, the polymeric layer being disposed on or about the surface layer.

FIG. 3C is a diagrammatic illustration of a cross section of a portion of a device comprising a surface layer and polymeric layer comprising therapeutic agent, pores, and therapeutic agent in the pores, the polymeric layer being disposed on or about the surface layer.

FIG. 4A is a diagrammatic illustration of a cross-section of a portion of a device comprising a surface layer, and intermediate layer disposed on or about the surface layer, and a polymeric layer comprising pores and therapeutic agent disposed in the pores, the polymeric layer being disposed on or about the intermediate layer.

FIG. 4B is a diagrammatic illustration of a cross-section of a portion of a device comprising a surface layer, and intermediate layer disposed on or about the surface layer, and a polymeric layer comprising therapeutic agent and pores disposed on or about the intermediate layer.

FIG. 4C is a diagrammatic illustration of a cross-section of a portion of a device comprising a surface layer, and intermediate layer disposed on or about the surface layer, and a polymeric layer disposed on or about the intermediate layer, the polymeric layer comprising therapeutic agent, pores, and therapeutic agent in the pores.

FIG. 5A is a diagrammatic illustration of a cross-section of a portion of a device comprising a surface layer comprising therapeutic agent and polymeric layer disposed on or about the surface layer, the polymeric layer comprising therapeutic agent, pores, and therapeutic agent in the pores.

FIG. 5B is a diagrammatic illustration of a cross-section of a portion of a device comprising a surface layer comprising therapeutic agent, an intermediate layer disposed on or about the surface layer, and polymeric layer disposed on or about the intermediate layer, the polymeric layer comprising therapeutic agent, pores, and therapeutic agent in the pores.

FIG. 5C is a diagrammatic illustration of a cross-section of a portion of a device comprising a surface layer comprising therapeutic agent, an intermediate layer comprising therapeutic agent disposed on or about the surface layer, and polymeric layer disposed on or about the intermediate layer, the polymeric layer comprising therapeutic agent, pores, and therapeutic agent in the pores.

FIG. 5D is a diagrammatic illustration of a cross-section of a portion of a device comprising a surface layer, an intermediate layer comprising therapeutic agent disposed on or about the surface layer, and polymeric layer disposed on or about the intermediate layer, the polymeric layer comprising therapeutic agent, pores, and therapeutic agent in the pores.

FIG. 6 is a photograph of a cross-section of a coated device according to an embodiment of the invention.

FIG. 7 is a graph showing release of dexamethasone from coated devices according to embodiments of the invention.

The drawings are not necessarily to scale. Like numbers refer to like parts or steps throughout the drawings.

### DETAILED DESCRIPTION

In the following description, reference is made to the accompanying drawings that form a part hereof, and in which are shown by way of illustration several specific embodiments of the invention. It is to be understood that other embodiments of the present invention are contemplated and may be made without departing from the scope or spirit of the present invention. The following detailed description, therefore, is not to be taken in a limiting sense.

Various embodiments of the present invention relate to extravascular implantable medical devices capable of eluting a therapeutic agent from a polymeric layer of the device when implanted in a patient. The polymeric layer comprises pores, which can serve as a means for increasing the rate of release of therapeutic agent from the device and/or as a means for increasing the amount of therapeutic agent that can be loaded on or in the device. The pores may also serve as a means for retaining therapeutic agent that may not otherwise be amenable to loading in the polymeric layer. Accordingly, various extravascular implantable devices comprising a porous polymer layer according to various embodiments of the invention may allow for finer control of release of therapeutic agent and increased loading ability of therapeutic agent to be eluted from the devices.

It should be understood that, as used herein “implanted medical device”, “implantable medical device”, and the like refer to medical devices that are to be at least partially placed within a patient's body. Typically, such devices, or portions thereof, are placed within the patient's body for a period of time for which it would be beneficial to have a therapeutic agent present on a surface of the device. For example, a medical device implanted in a patient's body for several hours or more constitutes an implantable medical device for the purposes of this disclosure.

Overview

Embodiments of the invention provide extravascular implantable devices comprising a polymeric layer for eluting a therapeutic agent after implantation in an extravascular location of a patient. Non-limiting examples of extravascular implantable medical devices include pulse generators, infusion pumps, defibrillators, pacemakers, catheters, leads, lead extensions, bone grafts, and the like. It will be understood that certain catheters, leads, and lead extensions may be implanted intravascularly. Catheters, leads, and lead extensions according to various embodiments of the invention include catheters, leads, and lead extensions having a stiffness outside the range of those used for intravascular purposes.

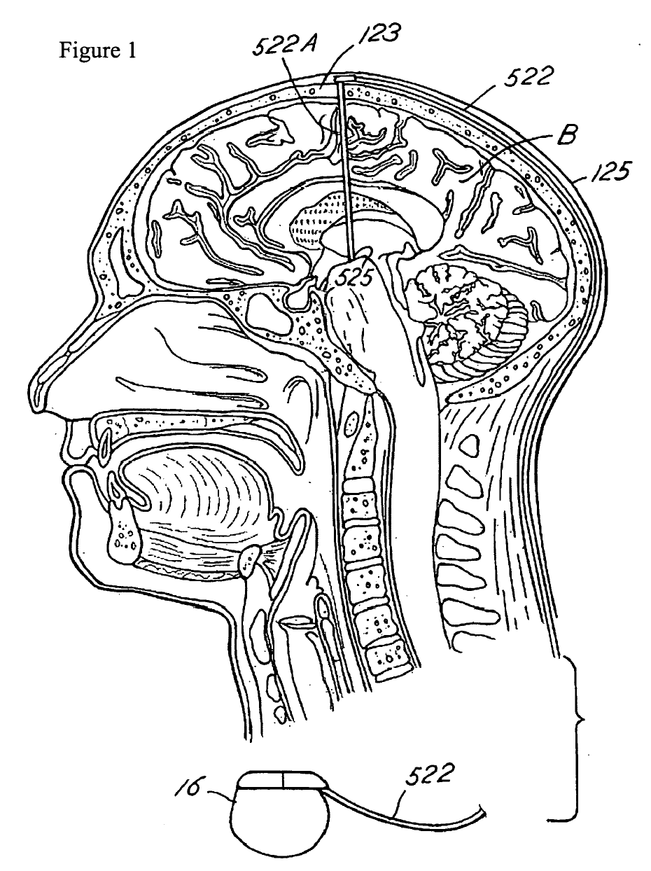

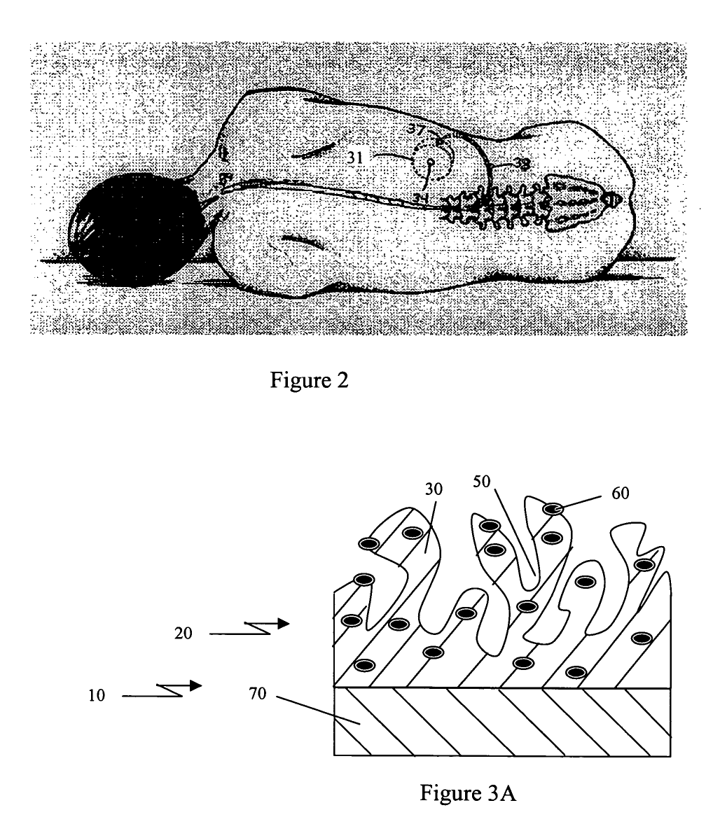

Any extravascular implantable device may be modified according to the teaching of the present disclosure. Non-limiting examples of extravascular implantable medical devices that may be modified to elute a therapeutic agent according to the teachings of the present disclosure are shown in FIGS. 1 and 2.

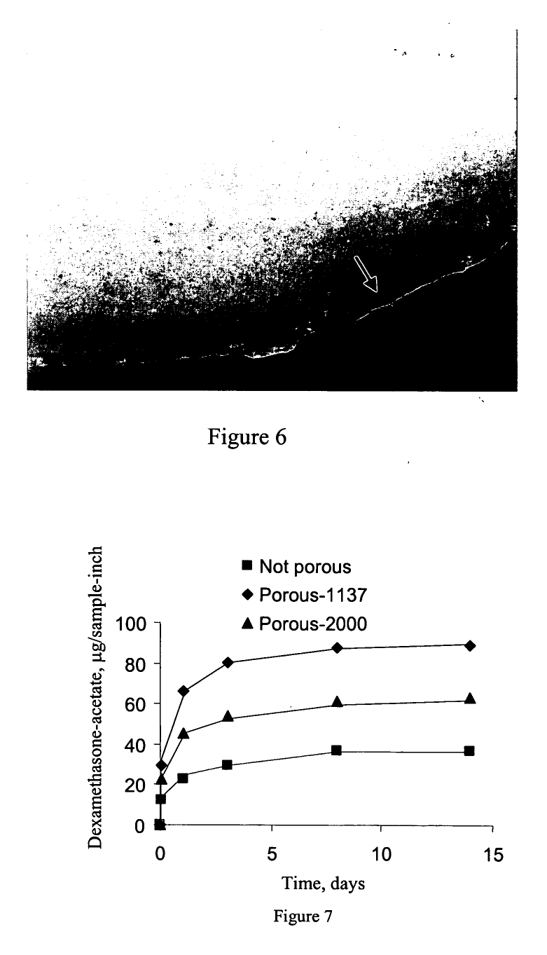

FIG. 1 depicts a neurostimulator system implanted in a patient. The system comprises an implantable pulse generator 16, a lead extension 522, a lead 522A, lead/lead extension connector 127, and at least one electrode positioned in proximity to the distal end of lead 522A. Pulse generator 16 is typically implanted subcutaneously in a patient, most typically in the abdomen or chest. However, it will be understood that pulse generator 16 may be implanted anywhere within a patient. Preferably the pulse generator 16 is implanted in a location that causes minimal discomfort to the patient and still allows for proper functioning. From the location of implantation of pulse generator 16, lead extension 522 is typically tunneled subcutaneously to a position in proximity to a target therapy site. In the embodiment shown in FIG. 1, the target therapy site is within the patient's brain B. However, it will be understood that the target therapy site may be any other location where a patient may benefit from electrical stimulation therapy, such as e.g. other regions of the CNS, including the spinal cord; and regions of the peripheral nervous system, including autonomic nerves and enteric nerves. Lead 522A is positioned such that one or more electrodes are in or in close proximity to the target therapy site. Lead 522A is typically connected to lead extension 522 through a connector 127. In the embodiment shown in FIG. 6, a hole is drilled through the patient's skull 123 and lead 522A is inserted through the hole into patient's brain B such that one or more electrodes are in or near the target site. A porous polymeric layer comprising a therapeutic agent according to the teachings of the present disclosure may be disposed on or about at least a portion of an external surface of one or more of pulse generator 16, lead extension 522, connector 127, and any other associated components (not shown).

Referring to FIG. 2, an infusion system implanted in a patient is shown. The infusion system comprises an implantable infusion pump 31 comprising a re-fill port 34 and a catheter connection port 37, and a catheter 38 connectable to the catheter connection port 37. Catheter comprises one or more infusion sites through which a drug housed in a reservoir of implantable pump 31 may be delivered to a target site of the patient. Typically, infusion pump 31 is implanted in a subcutaneous pocket in the patient as shown in FIG. 2. The pump 31 may be implanted in any medically acceptable location within the patient. Typically, pump 31 is implanted into the patient's abdomen. The catheter is then typically tunneled to a location such that one or more infusion site is placed at or near a target treatment site in the patient. In FIG. 2, the catheter 38 is introduced into the intrathecal space such that distal portion 39 of catheter resides within the patient's spinal canal. A porous polymeric layer comprising a therapeutic agent according to the teachings of the present disclosure may be disposed on or about at least a portion of any of one or more of implantable infusion pump 31, an external surface of catheter 38 located outside patient's spinal canal, and any other associated components (not shown).

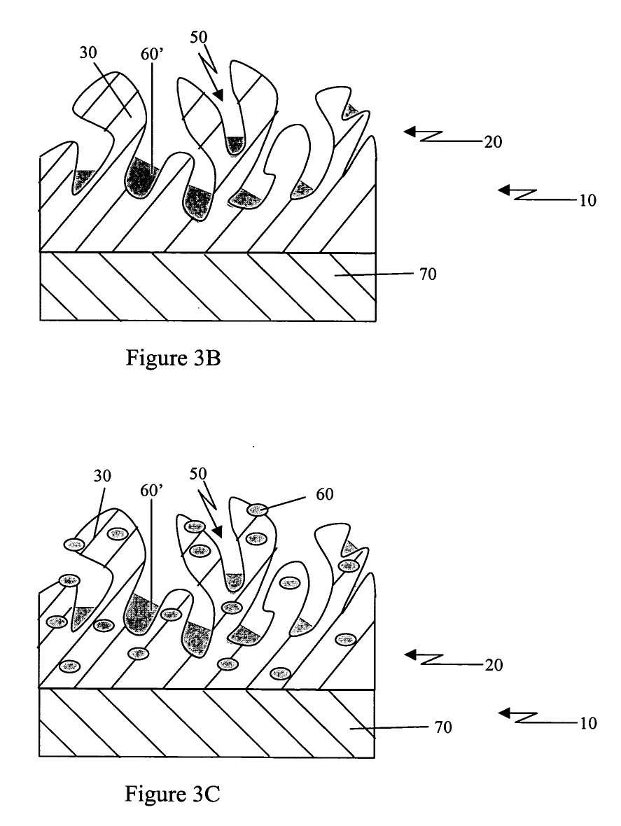

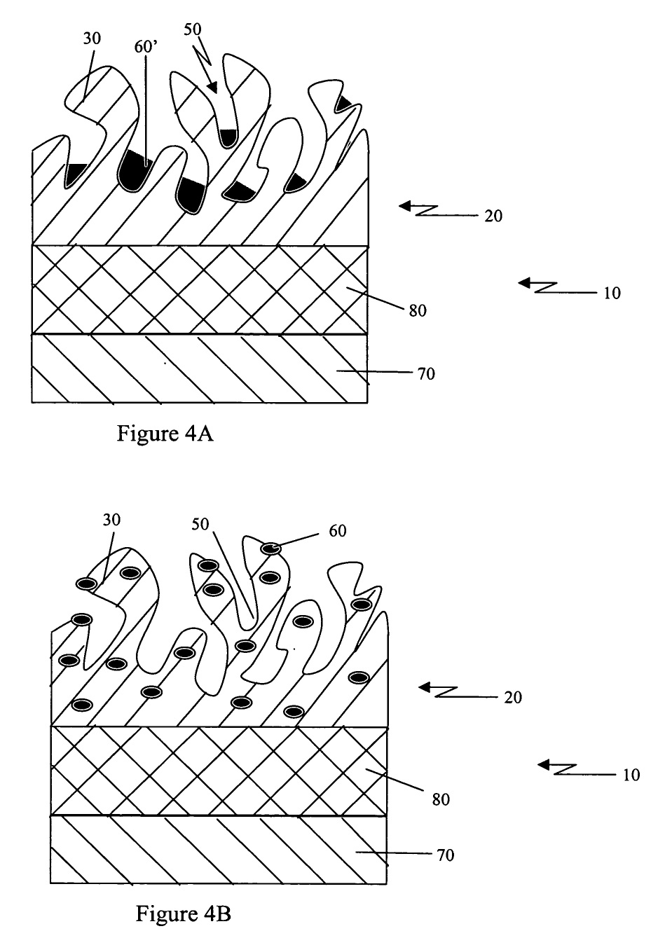

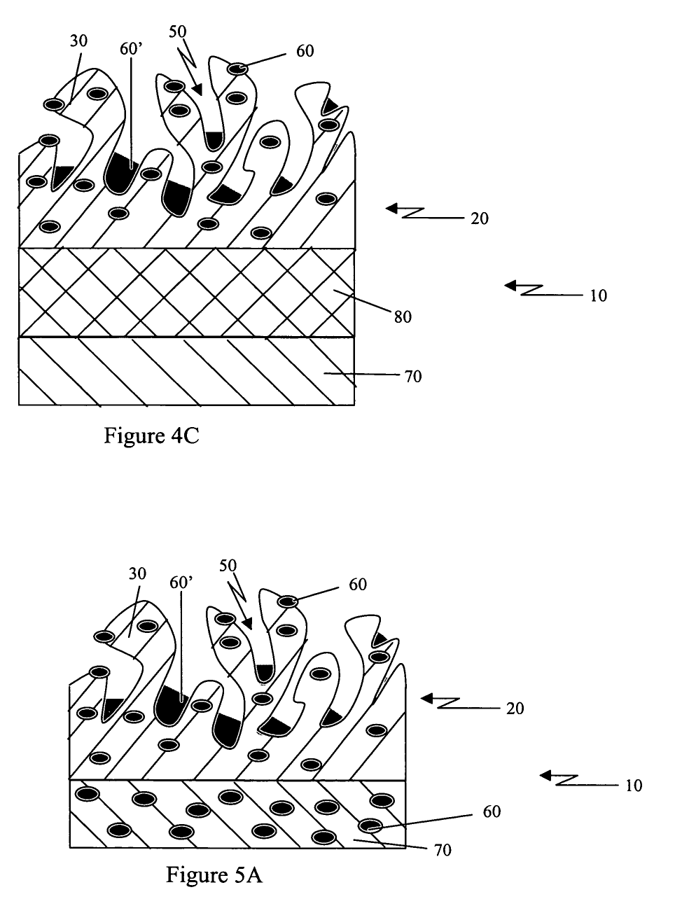

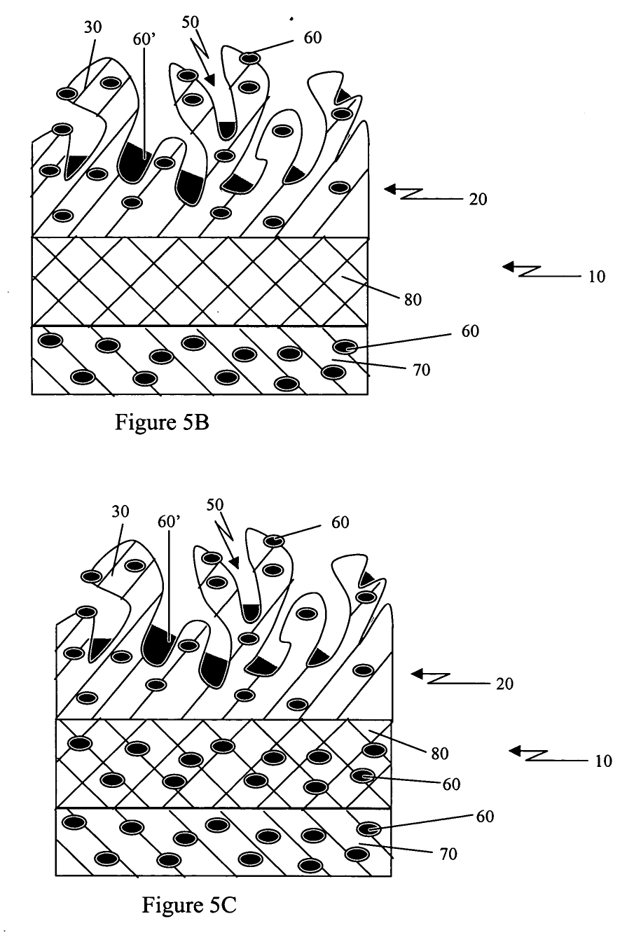

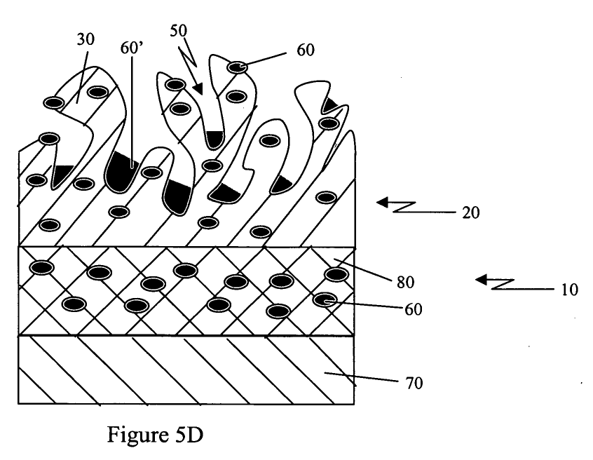

Examples of portions of extravascular implantable devices 10 according to various embodiments of the invention are shown in FIGS. 3-5. As shown in FIGS. 3A-3C and 5A, polymeric layer 20 may be disposed on surface layer 70. Alternatively, as illustrated in FIGS. 4A-4C and 5B-5D, an intermediate layer 80 may be disposed between polymeric layer 20 and surface layer 70. It will be understood that two, three, four, five, or more intermediate layers 80 may be disposed between polymeric layer 20 and surface layer 70. Intermediate layer may be formed of any material. Preferably, intermediate layer 80 is formed of biocompatible material. Intermediate layer 80 may comprise one or more polymers that may be the same or different from those of polymeric layer 20. One or more intermediate layer 80 may comprise a porous or non-porous polymeric material. Therapeutic agent 60 placed in a porous intermediate layer 20 (not shown) may be expected to be released into tissue more rapidly than if placed in a non-porous intermediate layer 20, as therapeutic agent 60 from an underlying porous layer should permeate through a porous polymer more rapidly than through a non-porous polymer. If an intermediate layer 80 is porous, therapeutic agent 60 may be disposed in pores (not shown) of the intermediate layer 80 and/or may be disposed in or on the polymeric matrix of the intermediate layer 80. Accordingly, the release profile of therapeutic agent 60 may be more finely controlled by selecting placement in pores 50, matrix 30 of porous polymeric material 20, and matrix or pores of underlying porous polymeric material. Therapeutic agent 60 may be disposed in or on surface layer 70 and/or intermediate layer 80, as shown in FIGS. 5A-5C.

As shown in FIG. 3C, polymeric layer 20 comprising polymeric matrix 30, therapeutic agent 60 in or on matrix 30, pores 50, and therapeutic agent 60′ disposed in pores 50, may be disposed on surface layer 70 of device 10. It will be understood that therapeutic agent 60 and therapeutic agent 60′ may be the same or different and may refer to a plurality of therapeutic agents. A configuration as depicted in FIG. 3C, may be desirable in many situations. For example, if therapeutic agent 60 or 60′ is incompatible with surface layer 70, polymeric layer 20 may serve as a buffer between surface layer 70 and therapeutic agent 60, 60′. If it is difficult to load sufficient quantities of therapeutic agent 60, 60′ on or in surface layer 70 or if it is difficult to control the release profile of therapeutic agent 60, 60′ from surface layer 70, polymeric layer 20 may serve as a means to load and control release of sufficient quantities of therapeutic agent 60, 60′. If loading therapeutic agent 60, 60′ in or on surface layer 70 would impair the integrity of device 10, polymeric layer 20 may serve as a means for maintaining the structural or functional integrity of surface layer 70 while still providing for release of therapeutic agent 60, 60′.

As shown in FIG. 3A, therapeutic agent 60 may be disposed in polymeric matrix 30 of polymeric layer 20. The presence of pores 50 in polymeric layer 20 may serve to facilitate release of therapeutic agent 60 from polymer layer after device 10 is implanted in an extravascular location of a patient. The release rate of therapeutic agent 60 from polymeric layer 20 may be controlled by varying the average size of pores 50 and the degree of porousity of polymeric layer 20. The presence of pores may also serve to facilitate tissue in-growth, thus bringing tissue to be treated with therapeutic agent 60 into closer proximity to therapeutic agent 60.

As shown in FIG. 3B, therapeutic agent 60′ is disposed in pores 50 of polymeric layer 20. Such a configuration may be desirable when therapeutic agent 60′ is difficult to introduce into polymeric matrix 30, such as with, e.g., large and or polar therapeutic agents 60′, which may be difficult to load into, e.g., silicone. Such a configuration may also be preferred when relatively rapid release of therapeutic agent 60′ from polymeric layer 20 is desired.

FIGS. 4A-4C show devices 10 in which an intermediate layer 80 is disposed between surface layer 70 and polymeric layer 20. The presence of intermediate layer(s) 80, may be desirable in many situations. For example, intermediate layer(s) 80 may serve as a buffer between potentially incompatible therapeutic agent 60, 60′ and surface layer 70 or potentially incompatible polymeric layer 20 and surface layer 70. Intermediate layer(s) 80 may serve to enhance the structural integrity of device 10. Further, as shown in FIGS. 5C and 5D, intermediate layer(s) 80 may serve as a means for loading and eluting therapeutic agent 60. The ability of intermediate layer(s) 80 to form a protective buffer, enhance integrity, or control release of therapeutic agent 60 will depend on the material from which intermediate layer(s) are formed, as well as the thickness and number of intermediate layers 80.

As shown in FIGS. 5A-5C, surface layer 70 of device 10 may serve as a means for loading therapeutic agent 60. Release of therapeutic agent 60 from surface layer 70 to tissue into which device 10 is implanted will likely occur more slowly than release from intermediate layer(s) 80 or polymeric layer 20. Thus, the release profile of therapeutic agent 60, 60′ may be controlled by the amount of therapeutic agent 60, 60′ in or on surface layer 70, intermediate layer(s) 80, polymeric matrix 30, and pores 50.

While not shown, it will be understood that a barrier layer, such as a polymer barrier, may be disposed on polymeric layer 20. Such a barrier layer may reduce the rate of release of therapeutic agent 60, 60′ from device 10 after implantation and may serve to hold therapeutic agent 60, 60′ in pores 50 during the implantation procedure. The extent to which barrier layer reduces the release rate of therapeutic agent 60, 60′ may depend upon the thickness of barrier layer, the porosity of barrier layer, and the material from which barrier layer is formed.

Polymeric Layer

Polymeric layer 20 may be formed of any material capable of releasing therapeutic agent 60, 60′ into tissue when placed in contact with the tissue. Preferably, polymeric layer 20 is acceptable for at least temporary use within a human body. Polymeric layer 20 is also preferably compatible with therapeutic agent 60, 60′.

Examples of commonly used materials that may be used to form polymeric layer 20 include organic polymers such as silicones, polyamines, polystyrene, polyurethane, acrylates, polysilanes, polysulfone, methoxysilanes, and the like. Other polymers that may be utilized include polyolefins, polyisobutylene and ethylene-alphaolefin copolymers; acrylic polymers and copolymers, ethylene-covinylacetate, polybutylmethacrylate; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; polycarbonates; polyoxymethylenes; polyimides; polyethers; epoxy resins; polyurethanes; rayon; rayon-triacetate; cellulose; cellulose acetate, cellulose butyrate; cellulose acetate butyrate; cellophane; cellulose nitrate; cellulose propionate; cellulose ethers; carboxymethyl cellulose; polyphenyleneoxide; and polytetrafluoroethylene (PTFE).

Polymeric layer 20 according to various embodiments of the invention may comprise a biodegradable polymeric material, such as synthetic or natural bioabsorbable polymers.

Synthetic bioabsorbable polymeric materials that can be used to form the coating layers include poly (L-lactic acid), polycaprolactone, poly(lactide-co-glycolide), poly(ethylene-vinyl acetate), poly(hydroxybutyrate-covalerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) such as PEO/PLA, polyalkylene oxalates, and polyphosphazenes. According to another exemplary embodiment, the polymeric materials can be natural bioabsorbable polymers such as, but not limited to, fibrin, fibrinogen, cellulose, starch, collagen, and hyaluronic acid.

Polymeric layer 20 may be designed to control the rate at which therapeutic agent 60, 60′is released, leached, or diffuses from the polymeric layer 20. As used herein, “release”, “leach”, “diffuse”, “elute” and the like are used interchangeably when referring to a therapeutic agent 60. 60′ with respect to polymeric layer 20, intermediate layer 80, or surface layer 70 of device 10. Any known or developed technology may be used to control the release rate. For example, a coating layer may be designed according to the teachings of WO/04026361, entitled “Controllable Drug Releasing Gradient Coating for Medical Devices.”

In an embodiment polymeric layer 20 is formed from a non-biodegradable polymeric material, such as silicone or polyurethane.

Polymeric layer 20 may be in the form of a tube, jacket, sheath, sleeve, cover, coating, or the like. Polymeric layer 20 may be extruded, molded, coated on surface layer 70 or intermediate layer 80, grafted onto surface layer 70 or intermediate layer 80, embedded within surface layer 70 or intermediate layer 80, adsorbed to surface layer 70 or intermediate layer 80, etc. Polymers of polymeric layer 20 may be porous, or may be made porous. Porous materials known in the art include those disclosed in U.S. Pat. No. 5,609,629 (Fearnot et al.) and U.S. Pat. No. 5,591,227 (Dinh et al.). Typically polymers are non-porous. However, non-porous polymers may be made porous through known or developed techniques, such as extruding with CO₂, by foaming the polymeric material prior to extrusion or coating, or introducing and then removing a porogen. Non-limiting examples of porogens include salts, such as sodium bicarbonate, gelatin beads, sugar crystals, polymeric microparticles, and the like. One or more porogen may be incorporated into a polymer prior to curing or setting. The polymer may then be cured or set, and the porogen may be extracted with an appropriate solvent. Pores 50 generated by such techniques or processes typically range in size from between about 0.01 μm to about 100 μm. The size and degree of porosity of polymeric material 20 may be controlled by the size and concentration of porogen used, the extent of mixing with gas or foaming, etc. Accordingly, the release profile of therapeutic agent 60, 60′ from polymeric layer 20 may be controlled by varying the conditions under which pores 50 are generated, as pore size and degree of porosity are related to release rate. Larger pore 50 size, e.g., between about 1 μm and about 100 μm or between about 10 μm to 50 μm may be preferred when more rapid release of therapeutic agent 60 from polymeric layer 20 is desired.

Depending upon the type of materials used to form polymeric layer 20, polymeric layer 20 can be applied to the surface layer 70 or intermediate layer 80 through any coating processes known or developed in the art. One method includes directly bonding polymeric layer 20 to surface layer 70 or underlying intermediate layer 80. By directly attaching a polymeric layer 20 to surface layer 70 or intermediate layer 80, covalent chemical bonding techniques may be utilized. Surfaces of surface layer 70 or intermediate layer 80 may possess chemical functional groups, such as carbonyl groups, primary amines, hydroxyl groups, or silane groups which will form strong, chemical bonds with similar groups on polymeric layer 20 utilized. In the absence of such chemical forming functional group, known techniques may be utilized to activate a material's surface before coupling the biological compound. Surface activation is a process of generating, or producing, reactive chemical functional groups using chemical or physical techniques such as, but not limited to, ionization, heating, photochemical activation, oxidizing acids, sintering, physical vapor deposition, chemical vapor deposition, and etching with strong organic solvents. Alternatively, polymeric layer 20 may be indirectly bound to surface layer 70 or intermediate layer 80 through intermolecular attractions such as ionic or Van der Waals forces. Of course, if polymeric layer 20 is in the form of a jacket, sheath, sleeve, cover, or the like, the chemical interaction between polymeric layer 20 and surface layer 70 or intermediate layer 80 may be minimal.

Therapeutic agent 60, 60′ may be incorporated into polymeric layer 20 in a variety of ways. For example, therapeutic agent 60, 60′ can be covalently grafted to a polymer of the polymeric layer 20, either alone or with a surface graft polymer. Alternatively, therapeutic agent 60, 60′ may be coated onto the surface of the polymer either alone or intermixed with an overcoating polymer. Therapeutic agent 60, 60′ may be physically blended with a polymer of a polymeric layer 20 as in a solid-solid solution. Therapeutic agent 60, 60′ may be impregnated into a polymer by swelling the polymer in a solution of the appropriate solvent. Any means of incorporating therapeutic agent 60, 60′ into or on a polymeric layer 20 may be used, provided that therapeutic agent 60, 60′ may be released, leached or diffuse from polymeric layer 20 on contact with bodily fluid or tissue.

A polymer of a polymeric layer 20 and a therapeutic agent 60, 60′ may be intimately mixed either by blending or using a solvent in which they are both soluble. This mixture can then be formed into the desired shape or coated onto an underlying structure of the medical device. One exemplary method includes adding one or more therapeutic agent 60, 60′ to a solvated polymer to form a therapeutic agent 60, 60′/polymer solution. The therapeutic agent 60, 60′/polymer solution can then be applied directly to the surface layer 70 or intermediate layer 80; for example, by either spraying or dip coating device 10. As the solvent dries or evaporates, the therapeutic agent 60, 60′/polymer coating is deposited on device 10. Furthermore, multiple applications can be used to ensure that the coating is generally uniform and a sufficient amount of therapeutic agent 60, 60′ has been applied to device 10.

Alternatively, an overcoating polymer, which may or may not be the same polymer that forms the primary polymer of surface layer 70 (it will be understood that in some embodiments the external surface layer 12 of device 10 is formed of a polymeric material and in other embodiments the external surface layer 12 of device 10 is from non-polymeric material, such as metallic material) or intermediate layer 80, and therapeutic agent 60, 60′ are intimately mixed, either by blending or using a solvent in which they are both soluble, and coated onto surface layer 70 or intermediate layer 80. Any overcoating polymer may be used, as long as the polymer is able to bond (either chemically or physically) to the polymer of an underlying layer of device 10.

In addition, a polymer of a polymeric layer 20 may be swelled with an appropriate solvent, allowing a therapeutic agent 60, 60′ to impregnate the polymer.

Therapeutic agent 60, 60′ may also be covalently grafted onto a polymer of a polymeric layer 20. This can be done with or without a surface graft polymer. Surface grafting can be initiated by corona discharge, UV irradiation, and ionizing radiation. Alternatively, the ceric ion method, previously disclosed in U.S. Pat. No. 5,229,172 (Cahalan et al.), may be used to initiate surface grafting.

Additional therapeutic agent 60′ may be added to pores 50 by any known or future developed technique or procedure. For example, additional therapeutic agent 60′ may be added to pores 50 using a technique or process as described above. In an embodiment, additional therapeutic agent 60′ is disposed in pores 50 by contacting pores with a mixture comprising a solvent and additional therapeutic agent 60′. The solvent may be removed, by e.g. evaporation, leaving additional therapeutic agent 60′ disposed in pores 50. The solvent may or may not be a solvent that allows penetration of additional therapeutic agent 60′ into polymeric matrix 30.

Therapeutic Agent

Any therapeutic agent 60, 60′ may be disposed in or on polymeric matrix 30, pores 50, surface layer 70, or intermediate layer 80. Therapeutic agent 60 disposed in or on surface layer 70 may be the same or different than therapeutic agent 60 disposed in or on intermediate layer, which may be the same or different than therapeutic agent 60 disposed in or on polymeric matrix 30, which may be the same or different than therapeutic agent 60′ disposed in pores. As used herein, “therapeutic agent 60” and “therapeutic agent 60′” may be used interchangeably and may refer to more than one therapeutic agent.

It will be understood that therapeutic agent 60 may be present in polymeric layer 20, intermediate layer 80 or surface layer 70 in a mixture with an additional material designed to control the release rate of therapeutic agent 60. Such a configuration may be particularly desirable when therapeutic agent 60 is disposed in pores 50 of polymeric layer 20. Such additional materials are known to those of skill in the art and include polymeric materials.

Because it may be desirable to treat or prevent infections and/or inflammation associated with implantation of a medical device 10, it may be desirable to dispose one or more anti-infective agent and/or one or more anti-inflammatory agent in, on, or about at least a portion of an external surface of device 10. In addition, in some circumstances it may be desirable to deliver a local anesthetic. Additional or other agents that may be disposed in or on polymeric matrix 30, pores 50, surface layer 70, or intermediate layer 80 will be readily evident to one of skill in the art. A brief summary of some non-limiting classes of therapeutic agents that may be used follows.

1. Anti-Infective Agents

Any anti-infective agent may be used in accordance with various embodiments of the invention. As used herein, “anti-infective agent” means an agent that kills or inhibits the growth of an infective organism, such as a microbe or a population of microbes. Anti-infective agents include antibiotics and antiseptics.

A. Antibiotic

Any antibiotic suitable for use in a human may be used in accordance with various embodiments of the invention. As used herein, “antibiotic” means an antibacterial agent. The antibacterial agent may have bateriostatic and/or bacteriocidal activities. Nonlimiting examples of classes of antibiotics that may be used include tetracyclines (e.g. minocycline), rifamycins (e.g. rifampin), macrolides (e.g. erythromycin), penicillins (e.g. nafcillin), cephalosporins (e.g. cefazolin), other beta-lactam antibiotics (e.g. imipenem, aztreonam), aminoglycosides (e.g. gentamicin), chloramphenicol, sufonamides (e.g. sulfamethoxazole), glycopeptides (e.g. vancomycin), quinolones (e.g. ciprofloxacin), fusidic acid, trimethoprim, metronidazole, clindamycin, mupirocin, polyenes (e.g. amphotericin B), azoles (e.g. fluconazole) and beta-lactam inhibitors (e.g. sulbactam). Nonlimiting examples of specific antibiotics that may be used include minocycline, rifampin, erythromycin, nafcillin, cefazolin, imipenem, aztreonam, gentamicin, sulfamethoxazole, vancomycin, ciprofloxacin, trimethoprim, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, enoxacin, fleroxacin, temafloxacin, tosufloxacin, clinafloxacin, sulbactam, clavulanic acid, amphotericin B, fluconazole, itraconazole, ketoconazole, and nystatin. Other examples of antibiotics, such as those listed in Sakamoto et al., U.S. Pat. No. 4,642,104, which is herein incorporated by reference in its entirety, may also be used. One of ordinary skill in the art will recognize other antibiotics that may be used.

In general, it is desirable that the selected antibiotic(s) kill or inhibit the growth of one or more bacteria that are associated with infection following surgical implantation of a medical device. Such bacteria are recognized by those of ordinary skill in the art and include Stapholcoccus aureus, Staphlococcus epidermis, and Escherichia coli. Preferably, the antibiotic(s) selected are effective against strains of bacteria that are resistant to one or more antibiotic.

To enhance the likelihood that bacteria will be killed or inhibited, it may be desirable to combine two or more antibiotics. It may also be desirable to combine one or more antibiotic with one or more antiseptic. It will be recognized by one of ordinary skill in the art that antimicrobial agents having different mechanisms of action and/or different spectrums of action may be most effective in achieving such an effect. In an embodiment, a combination of rifampin and micocycline is used. In an embodiment, a combination of rifampin and clindamycin is used.

B. Antiseptic

Any antiseptic suitable for use in a human may be used in accordance with various embodiments of the invention. As used herein, “antiseptic” means an agent capable of killing or inhibiting the growth of one or more of bacteria, fungi, or viruses. Antiseptic includes disinfectants. Nonlimiting examples of antiseptics include hexachlorophene, cationic bisiguamides (i.e. chlorhexidine, cyclohexidine) iodine and iodophores (i.e. povidone-iodine), para-chloro-meta-xylenol, triclosan, furan medical preparations (i.e. nitrofurantoin, nitrofurazone), methenamine, aldehydes (glutaraldehyde, formaldehyde), silver-containing compounds (silver sulfadiazene, silver metal, silver ion, silver nitrate, silver acetate, silver protein, silver lactate, silver picrate, silver sulfate), and alcohols. One of ordinary skill in the art will recognize other antiseptics that may be employed in accordance with this disclosure.

It is desirable that the antiseptic(s) selected kill or inhibit the growth of one or more microbe that are associated with infection following surgical implantation of a medical device. Such microbes are recognized by those of ordinary skill in the art and include Stapholcoccus aureus, Staphlococcus epidermis, Escherichia coli, Pseudomonus auruginosa, and Candidia.

To enhance the likelihood that microbes will be killed or inhibited, it may be desirable to combine two or more antiseptics. It may also be desirable to combine one or more antiseptics with one or more antibiotics. It will be recognized by one of ordinary skill in the art that antimicrobial agents having different mechanisms of action and/or different spectrums of action may be most effective in achieving such an effect. In a particular embodiment, a combination of chlorohexidine and silver sulfadiazine is used.

C. Antiviral

Any antiviral agent suitable for use in a human may be used in accordance with various embodiments of the invention. Nonlimiting examples of antiviral agents include acyclovir and acyclovir prodrugs, famcyclovir, zidovudine, didanosine, stavudine, lamivudine, zalcitabine, saquinavir, indinavir, ritonavir, n-docosanol, tromantadine and idoxuridine. One of ordinary skill in the art will recognize other antiviral agent that may be employed in accordance with this disclosure.

To enhance the likelihood that viruses will be killed or inhibited, it may be desirable to combine two or more antiviral agents. It may also be desirable to combine one or more antiseptics with one or more antiviral agent.

D. Anti-Fungal

Any anti-fungal agent suitable for use in a human may be used in accordance with various embodiments of the invention. Nonlimiting examples of anti-fungal agents include amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione. One of ordinary skill in the art will recognize other anti-fungal agents that may be employed in accordance with this disclosure.

To enhance the likelihood that viruses will be killed or inhibited, it may be desirable to combine two or more anti-fungal agents. It may also be desirable to combine one or more antiseptics with one or more anti-fungal agent.

2. Anti-Inflammatory Agents

Any anti-inflammatory agent suitable for use in a human may be used in accordance with various embodiments of the invention. Non-limiting examples of anti-inflammatory agents include steroids, such as cortisone, hydrocortisone, prednisone, dexamethasone, methyl-prednisilone, an derivatives thereof; and non-steroidal anti-inflammatory agents (NSAIDs). Non-limiting examples of NSAIDS include ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixerl, clonixin, meclofenamic acid, flunixin, coichicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate.

3. Local Anesthetics

Any local anesthetic agent suitable for use in a human may be used in accordance with various embodiments of the invention. Non-limiting examples of local anesthetics agents include lidocaine, prilocalne, mepivicaine, benzocaine, bupivicaine, amethocaine, lignocaine, cocaine, cinchocaine, dibucaine, etidocaine, procaine, veratridine (selective c-fiber blocker) and articaine.

4. Other Pharmacological Agents

Non-limiting examples of other pharmacological agents that may be used include: beta-radiation emitting isotopes, beclomethasone, fluorometholone, tranilast, ketoprofen, curcumin, cyclosporin A, deoxyspergualin, FK506, sulindac, myriocin, 2-aminochromone (U-86983), colchicines, pentosan, antisense oligonucleotides, mycophenolic acid, etoposide, actinomycin D, camptothecin, carmustine, methotrexate, adriamycin, mitomycin, cis-platinum, mitosis inhibitors, vinca alkaloids, tissue growth factor inhibitors, platinum compounds, cytotoxic inhibitors, alkylating agents, antimetabolite agents, tacrolimus, azathioprine, recombinant or monoclonal antibodies to interleukins, T-cells, B-cells, and receptors, bisantrene, retinoic acid, tamoxifen, compounds containing silver, doxorubicin, azacytidine, homoharringtonine, selenium compounds, superoxide-dismutase, interferons, heparin; Antineoplastic/antiangiogenic agents, such as antimetabolite agents, alkylating agents, cytotoxic antibiotics, vinca alkaloids, mitosis inhibitors, platinum compounds, tissue growth factor inhibitors, cisplatin and etoposide; Immunosuppressant agents, such as cyclosporine A, mycophenolic acid, tacrolimus, rapamycin, rapamycin analogue (ABT-578) produced by Abbott Laboratories, azathioprine, recombinant or monoclonal antibodies to interleukins, T-cells, B-cells and/or their receptors; Anticoagulents, such as heparin and chondroiten sulfate; Platelet inhibitors such as ticlopidine; Vasodilators such as cyclandelate, isoxsuprine, papaverine, dipyrimadole, isosorbide dinitrate, phentolamine, nicotinyl alcohol, co-dergocrine, nicotinic acid, glycerl trinitrate, pentaerythritol tetranitrate and xanthinol; Thrombolytic agents, such as stretokinase, urokinase and tissue plasminogin activators; Analgesics and antipyretics, such as the opioid analgesics such as buprenorphine, dextromoramide, dextropropoxyphene, fentanyl, alfentanil, sufentanil, hydromorphone, methadone, morphine, oxycodone, papaveretum, pentazocine, pethidine, phenopefidine, codeine dihydrocodeine; acetylsalicylic acid (aspirin), paracetamol, and phenazone; and Antiproliferative agents such as QP-2 (taxol), paclitaxel, rapamycin, tacrolimus, everolimus, actinomycin, methotrexate, angiopeptin, vincristine, mitocycin, statins, C-MYC antisense, sirolimus, restenASE, 2-chloro-deoxyadenosine, PCNA (proliferating cell nuclear antigent) ribozyme, batimastat, prolyl hydroxylase inhibitors, halofuginone, C-proteinase inhibitors, and probucol; and combinations and/or derivates thereof.

Surface Layer/Intermediate Layer/Barrier Layer

Surface layer 70 of device 10 may be made of any material of which a surface of a medical device is made. Preferably, surface layer 70 is formed of material acceptable for at least temporary use within a human body. In an embodiment, surface layer 70 is formed of a polymer or combination of polymers, such as described above for polymeric layer 20. In an embodiment, surface layer 70 is formed of a metallic material. Non-limiting examples of metallic material that may form surface layer include stainless steel, titanium, nickel, Nitinol, nickel-titanium, titanium alloys, and other alloys. When formed of a metallic material, surface layer 70 may be treated by, e.g., ionization, heating, photochemical activation, oxidizing acids, sintering, physical vapor deposition, chemical vapor deposition and/or etching with strong organic solvents, as discussed above, to facilitate disposing therapeutic agent 60, intermediate layer 80, or polymeric material 20 on surface layer 70.

Intermediate layer 80 and barrier layer may be made of any material. Preferably, intermediate layer 80 and barrier layer are made of material suitable for implantation in a human. Barrier layer and intermediate layer 80 may be made of polymeric material as described above for polymeric layer 20.

Various embodiments of the invention are disclosed. One skilled in the art will appreciate that the present invention can be practiced with embodiments other than those disclosed. The disclosed embodiments are presented for purposes of illustration and not limitation.

All printed publications, such as patents, technical papers, and brochures, and patent applications cited herein are hereby incorporated by reference herein, each in its respective entirety. As those of ordinary skill in the art will readily appreciate upon reading the description herein, at least some of the devices and methods disclosed in the patents and publications cited herein may be modified advantageously in accordance with the teachings of the present invention.

### EXAMPLE

The following example is provided to illustrate specific embodiments of the invention only, and should not be construed as limiting the scope of the invention.

### Example 1

Porous polymer retains more drug and increases initial burst release of drug release relative to non-porous polymer

Methods

Silicone tubing from a Medtronic Model 8831 catheter, having nominal dimensions of 0.050″ OD and 0.021″ ID, was cut into approximately 1 inch pieces. After cleaning in tetrahydrofuran (THF), tubing was dip coated with two solutions containing 15 g of either RTV 1137 or RTV 2000 (NuSil Technology, Carpinteria, Calif.) together with sodium bicarbonate salt (15 g) and THF solvent (45 g). After proper drying and curing, tubing was placed in deionized water to extract the sodium bicarbonate salt.

Lumens of original (non-porous) and porous samples were filled with RTV-1137 and cured to prevent drug loading into tubing lumens. Samples with blocked lumens were placed in 1% of dexamethasone acetate solution in acetone for 30 seconds followed by drying overnight at 37° C. Drug loaded samples were placed in 5 ml of PBS buffer and incubated under stirring conditions at 37° C. for 14 days. Released dexamethasone was determined by measuring light absorption at 240 nm.

Results

A photograph of the tubing cross-section for sample RTV-1137 is shown in FIG. 6, where arrows indicate the coating layer. Weight increases after dip coating and salt extraction were around 4.3 wt % and 7.5 wt % for RTV-1137 and RTV-2000, respectively.

Release curves of drug (dexamethasone) are given in FIG. 7, which shows that tubing comprising a porous layer was able to retain about three times more drug than tubing lacking a porous layer. In addition, the initial burst of drug release was greater in the tubing comprising a porous component relative to the non-porous tubing.

What is claimed is:

1. An implantable medical device configured for implantation in an extravascular location, comprising
a structural surface layer;
a polymeric layer comprising a polymeric matrix and a plurality of pores, the polymeric layer being disposed on or about the surface layer; and
a first therapeutic agent, wherein the first therapeutic agent is disposed in one or more of the plurality of pores.

2. The device of claim 1, wherein the first therapeutic agent is disposed in the one or more of the plurality of pores and in the polymeric matrix.

3. The device of claim 1, further comprising a second therapeutic agent different from the first therapeutic agent, the second therapeutic agent being disposed in the polymeric matrix.

4. The device of claim 3, further comprising a third therapeutic agent disposed in or on the structural surface layer, the third therapeutic agent being the same or different from the first therapeutic agent.

5. The device of claim 2, further comprising a third therapeutic agent disposed in or on the structural surface layer, the third therapeutic agent being the same or different from the first therapeutic agent.

6. The device of claim 1, further comprising a third therapeutic agent disposed in or on the structural surface layer, the third therapeutic agent being the same or different from the first therapeutic agent.

7. The device of claim 1, further comprising an intermediate layer disposed on or about the structural surface layer, wherein the polymeric layer is disposed on or about the intermediate layer.

8. The device of claim 7, further comprising a fourth therapeutic agent disposed in the intermediate layer, the fourth therapeutic agent being the same or different than first therapeutic agent.

9. The device of claim 8, wherein the intermediate layer comprises a porous polymer.

10. The device of claim 9, wherein the fourth therapeutic agent is disposed in the porous polymer, in one or more pores of the porous polymer, or in both the porous polymer and one or more pores of the porous polymer.

11. The device of claim 2, further comprising an intermediate layer disposed on or about the structural surface layer, wherein the polymeric layer is disposed on or about the intermediate layer.

12. The device of claim 11, further comprising a fourth therapeutic agent disposed in the intermediate layer, the fourth therapeutic agent being the same or different than that first therapeutic agent.

13. The device of claim 3, further comprising an intermediate layer disposed on or about the structural surface layer, wherein the polymeric layer is disposed on or about the intermediate layer.

14. The device of claim 13, further comprising a fourth therapeutic agent disposed in the intermediate layer, the fourth therapeutic agent being the same or different than that first therapeutic agent.

15. The device of claim 4, further comprising an intermediate layer disposed on or about the structural surface layer, wherein the polymeric layer is disposed on or about the intermediate layer.

16. The device of claim 15, further comprising a fourth therapeutic agent disposed in the intermediate layer, the fourth therapeutic agent being the same or different than that first therapeutic agent.

17. The device of claim 1, wherein the average size of the pores is in the range of between about 1 μm and 100 μm.

18. The device of claim 1, wherein the structural surface layer comprises a polymer.

19. The device of claim 18, wherein the polymer is silicone.

20. The device of claim 18, wherein the polymer is polyurethane.

21. The device of claim 18, wherein the device is a catheter.

22. The device of claim 18, wherein the device is a lead.

23. The device of claim 18, wherein the device is a lead extension.

24. The device of claim 1, wherein the structural surface layer comprises a metallic material.

25. The device of claim 24, wherein the metallic material is titanium.

26. The device of claim 24, wherein the device is an implantable pulse generator.

27. The device of claim 24, wherein the device is an implantable infusion pump.

28. The device of claim 1, wherein the first therapeutic agent is selected from the group consisting of an anti-infective agent, an anti-inflammatory agent, and a local anesthetic.

29. The device of claim 1, wherein the first therapeutic agent is selected from the group consisting of minocycline, rifampin, chlorhexidine, clindamycin, and a silver-containing compound.

30. The device of claim 29, wherein the first therapeutic agent is minocycline.

31. The device of claim 29, wherein the first therapeutic agent is rifampin.

32. The device of claim 29, wherein the first therapeutic agent is chlorhexidine.

33. The device of claim 29, wherein the first therapeutic agent is silver sulfadiazine.

34. The device of claim 29, wherein the first therapeutic agent is clindamycin.

35. The device of claim 2, wherein the first therapeutic agent is selected from the group consisting of an anti-infective agent, an anti-inflammatory agent, and a local anesthetic.

36. The device of claim 2, wherein the first therapeutic agent is selected from the group consisting of minocycline, rifampin, chlorhexidine, clindamycin, and a silver-containing compound.

37. The device of claim 3, wherein the second therapeutic agent is selected from the group consisting of an anti-infective agent, an anti-inflammatory agent, and a local anesthetic.

38. The device of claim 4, wherein the third therapeutic agent is selected from the group consisting of an anti-infective agent, an anti-inflammatory agent, and a local anesthetic.

39. The device of claim 8, wherein the fourth therapeutic agent is selected from the group consisting of an anti-infective agent, an anti-inflammatory agent, and a local anesthetic.
